(19) United States
(12) Patent Application Publication
(10) Pub. No.: US 2005/0039270 A1
(43) Pub. Date: Feb. 24, 2005

(54) Use of polycarboxylic acids and salts thereof as complexing agents in oxidizing compositions for dyeing, bleaching or permanently reshaping keratin fibres

(75) Inventors: Frederic Legrand, Courbevoie (FR); Jean-Marie Millequant, Saint-Maur-des-Fosses (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL S.A., Paris (FR)

(21) Appl. No.: 10/808,694

(22) Filed: Mar. 25, 2004

Related U.S. Application Data
(60) Provisional application No. 60/461,983, filed on Apr. 11, 2003.

(30) Foreign Application Priority Data
Mar. 28, 2003 (FR) 0350078
Mar. 25, 2003 (FR) 0350064

(51) Int. Cl. A61K 7/13

(52) U.S. Cl. 8/405

(57) ABSTRACT

The present invention relates to a ready-to-use oxidizing composition for bleaching or permanently reshaping keratin fibres. The oxidizing composition comprises polycarboxylic acids and salts thereof as complexing agents. The invention also relates to a method of using a oxidizing composition and a kit for bleaching or permanently reshaping keratin fibres.

### TECHNICAL FIELD

The present invention relates to the use of particular polycarboxylic acids and salts thereof as complexing agents in oxidizing compositions for dyeing, bleaching or permanently reshaping keratin fibres, in particular human keratin fibres and more especially the hair.

The invention also relates to oxidizing compositions for dyeing, bleaching or permanently reshaping keratin fibres, which contain such complexing agents, and also to processes and devices or “kits” for dyeing, bleaching or permanently reshaping keratin fibres.

### PRIOR ART

It is known practice, to dye keratin fibres, such as human hair, to use dye compositions containing oxidation dye precursors, in particular ortho- or para-phenylenediamines, ortho- or para-aminophenols, and heterocyclic bases, which are generally known as oxidation bases. Oxidation dye precursors are colourless or weakly coloured precursors, which, when combined with oxidizing products, can give rise to'coloured compounds and dyes via a process of oxidative condensation. The shades obtained with these oxidation bases can be varied by combining them with couplers or coloration modifiers, these agents being chosen from aromatic meta-diamines, meta-amino-phenols, meta-diphenols and certain heterocyclic compounds.

The coloration generated by the application to the fibres to be coloured of dye compositions in the presence of oxidizing products may be for the purpose of colouring grey hair, but also for modifying the colour of natural or dyed hair. In the latter case, it is necessary, in particular when the desired colour is several tones below the initial colour of the hair to be dyed, to bleach the hair to be dyed prior to dyeing it.

There is also a need to lighten, and thus to bleach, the hair without, however, wishing to recolour it.

Two types of composition are generally used to bleach keratin fibres: compositions that are “oxidizing”, since they contain one or more agents capable of oxidizing the melanin of the hair and thus of dissolving it in order to totally or partially remove it, and compositions that are, in contrast, “reducing”, since they contain one or more reducing agents such as ascorbic acid or thiols, the latter compositions being intended more especially for bleaching hair that has previously been dyed with exogenous pigments.

As regards permanent reshaping, it is common practice to apply to hair that has been placed under tension beforehand, for example using rollers if the desired reshaping is curls, a composition containing one or more reducing agents so as to induce the opening of the disulphide bridges formed by the cysteine residues of the hair keratin, and then, generally after rinsing, to reoxidize the hair in order to fix its reshaping, by applying an oxidizing composition.

As regards the oxidizing compositions that are necessary for performing the fixing step, use is usually made, in practice, of compositions based on aqueous hydrogen peroxide solution, sodium bromate or persalts, for instance sodium perborate.

Whether they are intended for dyeing, bleaching or permanent reshaping, the oxidizing compositions contain, in principle, a complexing agent intended to complex the metal cations that may be present in trace amounts in these compositions, and also those that may be present on the hair, originating from the ambient air, from the water with which this hair has been washed, or shampoos or other hair products with which the hair has been treated.

Specifically, it is very important to neutralize these metal cations, since they are capable of catalysing oxidation reactions on the hair fibres, and of doing so in an uncontrolled manner, which may be reflected by severe adverse effects such as breaking of the hair or burning of the scalp.

The complexing agents that are currently the most commonly used in oxidizing compositions for dyeing, bleaching or permanently reshaping keratin fibres are ethylenediaminetetraacetic acid (EDTA) and derivatives thereof, for instance diethylenetriaminepentaacetic acid (DPTA), generally in weight proportions of about 0.1% to 1%.

However, in the context of its research, the Applicant has found that EDTA and derivatives thereof have insufficient properties in compositions of this type. These findings, which are corroborated by the results obtained by other research teams, justify the search for novel complexing agents.

A complexing agent intended to form part of the constitution of oxidizing compositions for dyeing, bleaching or permanently reshaping keratin fibres should satisfy many requirements. Specifically, besides the fact that it should have high complexing power with respect to metals so as to remove, or at the very least minimize, the risk of catalysis of oxidation reactions on the keratin fibres by the traces of metals possibly present in these compositions and on these fibres, it should be compatible, and especially should not react, with the other constituents, and in particular with the oxidizing agent(s). It should also be stable in solution, since, specifically, the oxidizing compositions are generally applied to the keratin fibres in the form of solutions. It should also be entirely harmless with respect to these fibres and to the skin, and especially should have no allergenic nature.

It is also desirable, out of concern for the environment, for it to be biodegradable, and for its production cost or purchase cost to allow it to be used in compositions intended to be sold not only to professionals but also in mass distribution.

Now, after extensive research conducted in this matter, the Applicant has discovered that, surprisingly, certain polyacids and salts thereof are capable of satisfying all these requirements and consequently of representing complexing agents of choice in oxidizing compositions for dyeing, bleaching and permanently reshaping keratin fibres.

This discovery forms the basis of the invention.

### DESCRIPTION OF THE INVENTION

Thus, a first subject of the invention is the use, as an agent for complexing metal cations present in an oxidizing composition, comprising at least one oxidizing agent, for bleaching, dyeing or permanently reshaping keratin fibres, of at least one compound corresponding to formula (I) below:
R—N— (CH(R′)CO₂X)₂ (I)
in which:
R represents a hydrogen atom or a group —CH(CO₂X)—(CH₂)₂CO₂X, —CH(CH₃)—CO₂X or —(CH₂)₂—N(COR″)—CH₂—CO₂X;
R′ represents a group —CH₂CO₂X when R represents a hydrogen atom, whereas R′ represents a hydrogen atom when R is other than a hydrogen atom; and
R″ represents a linear or branched alkyl group containing from 1 to 30 carbon atoms, or a cycloalkyl group containing from 3 to 30 carbon atoms;
X represents a hydrogen atom or a monovalent or divalent cation derived from an alkali metal, from an alkaline-earth metal, from a transition metal or from an organic amine, or an ammonium cation.

Thus, the complexing agents used in the context of the invention correspond to polycarboxylic acid compounds and to the corresponding carboxylates.

More specifically, the complexing agents correspond to:
compounds comprising four carboxylic acid or carboxylate functions, when R represents a hydrogen atom and R′ represents a group —CH₂—CO₂X, or when R represents a group —CH(CO₂X)—(CH₂)₂—CO₂X and R′ represents a hydrogen atom;
compounds comprising three carboxylic acid or carboxylate functions, when R represents a group —CH(CH₃)—CO₂X and R′ represents a hydrogen atom, or when R represents a group —(CH₂)₂—N(COR″)—CH₂—CO₂X and R′ represents a hydrogen atom.

In accordance with the invention, when the compound(s) of formula (I) is(are) carboxylates, then the monovalent or divalent cation is preferably chosen from the group consisting of monovalent alkali metal cations, divalent alkaline-earth metal cations, divalent transition metal cations and monovalent cations derived from organic amines or from ammonium cations.

Examples of alkali metal cations that may especially be mentioned include sodium (Na⁺) and potassium (K⁺), while examples of alkaline-earth metal cations that may especially be mentioned include calcium (Ca²⁺) and magnesium (Mg²⁺).

For the purpose of the present invention, the term “transition metal” means a metal comprising an incomplete d subshell, more particularly in oxidation state II, such as cobalt (Co²⁺), iron (Fe²⁺), manganese (Mn²⁺), zinc (Zn²⁺) and copper (Cu²⁺).

As regards the organic amine monovalent cations, mention may be made of primary, secondary or tertiary amine cations or alternatively alkanolamine cations.

Said amines contain one or more radicals, which may be identical or different, of linear or branched C₁ to C₂₀ alkyl type, optionally comprising a heteroatom such as oxygen.

As regards the quaternary ammonium monovalent cations, these comprise three radicals, which may be identical or different, chosen from hydrogen and a linear or branched C₁ to C₂₀ alkyl radical, optionally comprising a heteroatom such as oxygen.

In accordance with the invention, when the compound(s) of formula (I) is (are) carboxylates, then the monovalent or divalent cation is preferably chosen from the group consisting of alkali metal cations, alkaline-earth metal cations and divalent transition metal cations, and cations derived from organic amines or from ammonium.

The compound(s) of formula (I) is (are) preferably chosen from the group consisting of methylglycinediacetic acid, N-lauroyl-N,N′,N′-ethylenediaminetriacetic acid, iminodisuccinic acid and N,N-dicarboxymethyl-L-glutamic acid, the alkali metal salts thereof, the alkaline-earth metal salts thereof, the transition metal salts thereof, the organic amine salts thereof and the ammonium salts thereof, and mixtures thereof.

Methylglycinediacetic acid, N-lauroyl-N,N′,N′-ethylenediamine-tiacetic acid, iminodisuccinic acid and N,N-dicarboxymethyl-L-glutamic acid and, and the salts thereof, are respectively represented by formulae (II), (III), (IV) and (V) below: 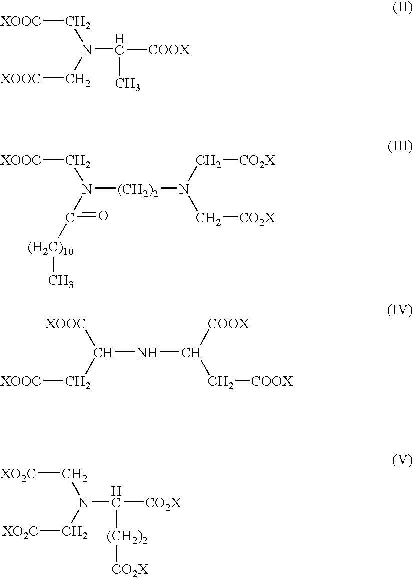
in which X is as defined above, X preferably corresponding to H or Na.

These compounds are especially available from the companies BASF, Dow Chemical, Hampshire, Bayer and Showa Denko.

Methylglycinediacetic acid and the sodium salts thereof are more particularly preferred.

Preferably, the compound(s) of formula (I) represent(s) from 0.001% to 10% by weight and better still from 0.001% to 5% by weight relative to the total weight of the oxidizing composition. The weight percentages are expressed relative to the acid form of the compound(s) of formula (I).

In accordance with the invention, the oxidizing composition comprises one or more oxidizing agents, which may be chosen from the oxidizing agents conventionally used in cosmetic compositions for dyeing, bleaching and permanently reshaping keratin fibres. Among the oxidizing agents conventionally used that may be mentioned are hydrogen peroxide, urea peroxide, alkali metal bromates, persalts such as perborates, percarbonates and persulphates, and peracids.

According to the invention, it is particularly advantageous to use as complexing agents methylglycinediacetic acid or iminodisuccinic acid, optionally in the form of salts in combination with aqueous hydrogen peroxide solution.

According to the invention, the oxidizing composition may comprise, in addition to the compound(s) of formula (I) and the oxidizing agent(s), one or more compounds chosen from:
(A) cationic or amphoteric conditioning polymers;
(B) nonionic, anionic, cationic or amphoteric amphiphilic polymers comprising at least one hydrophobic chain:
(C) anionic, nonionic, cationic, amphoteric or zwitterionic surfactants;
(D) rheology modifiers other than the polymers (B);
(E) pH modifiers (5);
(F) solvents (7).
   (A) Cationic or Amphoteric Conditioning Polymers:

For the purposes of the present invention, the term “cationic conditioning polymer” means any polymer which comprises cationic groups or groups that can be ionized into cationic groups and which can improve the cosmetic properties of keratin fibres, in particular the disentangling, the softness, the sheen and the volume.

The cationic or amphoteric conditioning polymers that are suitable are advantageously chosen from those already known per se as improving the cosmetic properties of the hair, that is to say, especially, those described in patents and patent applications EP 337 354, FR 2 270 846, FR 2 383 660, FR 2 598 611, FR 2 470 596, FR 2 519 863, FR 2 788 974 and FR 2 788 976 for a list of these compounds.

However, more specific examples of cationic conditioning polymers that may especially be mentioned include cationic polymers comprising at least primary, secondary, tertiary and/or quaternary amine groups, which either may form part of the main polymer chain, or may be borne by a side substituent directly attached thereto.

Thus, mention may be made of:
(1) copolymers of acrylamide and of dimethylaminoethyl methacrylate quaternized with dimethylsulphate or with a dimethylhalide (Hercofloc from Hercules); copolymers of acrylamide and of methacryloyloxyethyltrimethylammonium chloride (Bina Quat P 100 from Ciba Geigy); the copolymer of acrylamide and of methacryloyloxyethyltrimethylammonium methosulphate (Reten from Hercules); quaternized or non-quaternized vinylpyrrolidone/dialkylaminoalkyl acrylate or methacrylate copolymers (Gafquat range from ISP; Copolymer 845, 958 and 937 from ISP); dimethylaminoethyl methacrylate/vinylcaprolactam/vinylpyrrolidone terpolymers (Gaffix VC 713 from ISP); vinylpyrrolidone/methacrylamidopropyldimethylamine copolymers (Styleze CC 10 from ISP); vinylpyrrolidone/dimethylaminopropylmethacrylamide quaternized copolymers (Gafquat HS 100 from ISP);
(2) Cellulose ether derivatives comprising quaternary ammonium groups, as described in FR 1 492 597. These polymers are also defined in the CTFA dictionary as quaternary ammoniums of hydroxyethylcellulose that has reacted with an epoxide substituted with a trimethylammonium group;
(3) Cationic cellulose derivatives such as copolymers of cellulose or cellulose derivatives grafted with a water-soluble quaternary ammonium monomer, described especially in U.S. Pat. No. 4,131,576, such as hydroxyalkylcelluloses, for instance hydroxymethylcellulose, hydroxyethylcellulose or hydroxypropylcellulose grafted especially with a methacryloylethyltrimethylammonium, methacrylamidopropyltrimethylammonium or dimethyldiallylammonium salt;
(4) The cationic polysaccharides described more particularly in patents U.S. Pat. No. 3,589,578 and U.S. Pat. No. 4,031,307, such as guar gums containing trialkylammonium cationic groups. Guar gums modified with a salt, for instance the chloride, especially 2,3-epoxypropyltrimethylammonium chloride, are used for example;
(5) Polymers consisting of piperazinyl units and of divalent alkylene or hydroxyalkylene radicals containing straight or branched chains, optionally interrupted with oxygen, sulphur or nitrogen atoms or with aromatic or heterocyclic groups, and also the oxidation and/or quaternization products of these polymers. Such polymers are described especially in FR 2 162 025 and FR 2 280 361;
(6) Water-soluble polyaminoamides prepared in particular by polycondensation of an acidic compound with a polyamine, which are optionally crosslinked, optionally alkylated, or, if they comprise one or more tertiary amine functions, quaternized. These polymers are described especially in FR 2 252 840 and FR 2 368 508;
(7) Polyaminoamide derivatives resulting from the condensation of polyalkylene polyamines with polycarboxylic acids followed by an alkylation with difunctional agents. Examples that may be mentioned include adipic acid-dialkylaminohydroxyalkyldialkylenetriamine polymers in which the alkyl radical is C₁-C₄. Such polymers are described especially in FR 1 583 363;
(8) Polymers obtained by reacting a polyalkylene polyamine comprising two primary amine groups and at least one secondary amine group with a dicarboxylic acid chosen from diglycolic acid and saturated C₃-C₈ aliphatic dicarboxylic acids, and then with epichlorohydrin. Such polymers are described especially in U.S. Pat. No. 3,227,615 and U.S. Pat. No. 2,961,347;
(9) Cyclopolymers of alkyldiallylamine or of dialkyldiallylammonium, in homopolymer or copolymer form, as described in FR 2 080 759 and in its Certificate of Addition No. 2 190 406;
(10) Diquaternary ammonium polymers as described in FR 2 320 330, FR 2 270 846, FR 2 316 271, FR 2 336 434, FR 2 413 907, U.S. Pat. No. 2,273,780, U.S. Pat. No. 2,375,853, U.S. Pat. No. 2,388,614, U.S. Pat. No. 2,454,547, U.S. Pat. No. 3,206,462, U.S. Pat. No. 2,261,002, U.S. Pat. No. 2,271,378, U.S. Pat. No. 3,874,870, U.S. Pat. No. 4,001,432, U.S. Pat. No. 3,929,990, U.S. Pat. No. 3,966,904, U.S. Pat. No. 4,005,193, U.S. Pat. No. 4,025,617, U.S. Pat. No. 4,025,627, U.S. Pat. No. 4,025,653, U.S. Pat. No. 4,026,945 and U.S. Pat. No. 4,027,020.

Mention may be made, for example, of polymers comprising the following repeating units: 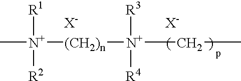
in which the radicals R¹, R², R³ and R⁴ which may be identical or different, denote a C₁-C₄ alkyl or hydroxyalkyl radical, n and p are integers ranging from 2 to 20 and X⁻ is an anion derived from a mineral or organic acid;
(11) Poly(quaternary ammonium) polymers consisting of repeating units of formula: 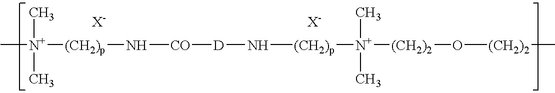
   in which p denotes an integer ranging from 1 to 6 approximately, D may be nothing or may represent a group —(CH₂)ᵣ—CO— in which r denotes a number equal to 4 or to 7, and X⁻ is an anion. Such polymers may be prepared according to the processes described in U.S. Pat. No. 4,157,388, U.S. Pat. No. 4,702,906, U.S. Pat. No. 4,719,282 and EP 122 324;
(12) Quaternary polymers of vinylpyrrolidone and of vinylimidazole;
(13) Polyamines of the polyethylene glycol (15) Tallow polyamine type (CTFA dictionary name);
(14) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethylammonium chloride copolymer (20/80 by weight) in the form of a dispersion containing 50% by weight of said copolymer in mineral oil (Salcare SC 92 from Ciba) can be used more particularly. A crosslinked methacryloyloxyethyltrimethylammonium chloride homopolymer containing about 50% by weight of the homopolymer in mineral oil or in a liquid ester (Salcare® SC 95, SC 96 from Ciba) can also be used.

Other cationic conditioning polymers that can be used in the context of the invention are polyalkyleneimines, in particular polyethyleneimines, polymers containing vinylpyridine or vinylpyridinium units, condensates of polyamines and of epichlorohydrin, quaternary polyureylenes and chitin derivatives.

The amphoteric conditioning polymer(s) that may be present in the oxidizing composition may themselves be chosen especially from polymers comprising units K and M randomly distributed in the polymer chain, in which K denotes a unit derived from a monomer comprising at least one basic nitrogen atom and M denotes a unit derived from an acidic monomer comprising one or more carboxylic or sulphonic groups; alternatively K and M may denote groups derived from zwitterionic carboxybetaine or sulphobetaine monomers; alternatively, K and M denote a cationic polymer chain comprising primary, secondary, tertiary or quaternary amine groups, in which at least one of the amine groups bears a carboxylic or sulphonic group linked via a hydrocarbon-based radical; alternatively K and M form part of a chain of a polymer containing an α,β-dicarboxylic ethylene unit in which one of the carboxylic groups has reacted with a polyamine comprising one or more primary or secondary amine groups.

The amphoteric conditioning polymers corresponding to the above definition that are more particularly preferred are chosen from the following polymers:
(1) polymers resulting from the copolymerization of a monomer derived from a vinyl compound bearing a carboxylic group such as, more particularly, (meth)acrylic acid, maleic acid, α-chloroacrylic acid, or else a dialkyldiallylammonium salt such as dimethyldiallylammonium chloride, and a basic monomer derived from a substituted vinyl compound containing at least one basic atom, such as, more particularly, dialkylaminoalkyl methacrylate and acrylate, dialkylaminoalkylmethacrylamide and -acrylamide, as described in U.S. Pat. No. 3,836,537. Mention may also be made of the sodium acrylate/acrylamidopropyltrimethylammonium chloride copolymer (Polyquart KE 3033 from Cognis) and the acrylic acid/dimethyldiallylammonium chloride copolymer (Merquat 280, 295, Plus 3330, from Nalco);
(2) polymers containing units derived from a) at least one monomer chosen from (meth)acrylamides substituted on the nitrogen with an alkyl radical, in particular C₂-C₁₂, b) at least one acidic monomer containing one or more reactive carboxylic groups (for example (meth)acrylic acid, crotonic acid or itaconic acid, and monoesters of maleic or fumaric acids or anhydrides), and c) at least one basic monomer such as esters containing primary, secondary, tertiary and quaternary amine substituents of (meth)acrylic acid, fumaric acid or maleic acid, and the product of quaternization of dimethylaminoethyl methacrylate with dimethyl or diethyl sulphate. Octylacrylamide/acrylate/butylaminoethyl methacrylate copolymers (Amphomer or Lovocryl 47 by the company National Starch) are particularly used.
(3) Crosslinked and partially or totally alkylated polyaminoamides, derived from polyaminoamides of general formula —[CO—R⁵—CO-Z]— in which R⁵ is a divalent radical derived from a saturated or unsaturated dicarboxylic acid (for example adipic acid, 2,2,4-trimethyladipic acid, 2,4,4-trimethyladipic acid, terephthalic acid or itaconic acid), from an unsaturated monocarboxylic acid (for instance (meth)acrylic acid), from a C₁-C₆ alkyl ester of the abovementioned acids or from a radical derived from the addition of one of these acids to a bis-primary or bis-secondary amine, and Z denotes a radical of a bis-primary, mono- or bis-secondary polyalkylene-polyamine. Preferably, Z represents between 60 and 100 mol %, the radical —NH—[(CH₂)ₓ—NH]ₚ— with x=2 and p=2 or 3, or x=3 and p=2; this radical is derived from diethylenetriamine, from triethylenetetramine or from dipropylenetriamine; between 0 and 40 mol % the above radical, in which x=2 and p=1 and which is derived from ethylenediamine, or the radical derived from piperazine —N[CH₂CH₂]₂N—; between 0 and 20 mol %, the radical —NH—(CH₂)₆—NH— derived from hexamethylenediamine. The crosslinking agent for these polymers is a difunctional agent chosen from epihalohydrins, diepoxides, dianhydrides and bis-unsaturated derivatives, and alkylated by the action of acrylic acid, chloroacetic acid or an alkane sultone or the alkali metal salts thereof;
(4) Polymers comprising at least zwitterionic units, for instance the butyl methacrylate/dimethylcarboxymethylammonioethyl methacrylate copolymer (Diaformer Z301 from Sandoz);
(5) Polymers derived from chitosan comprising monomer units corresponding to formulae (I), (II) and (III) below: 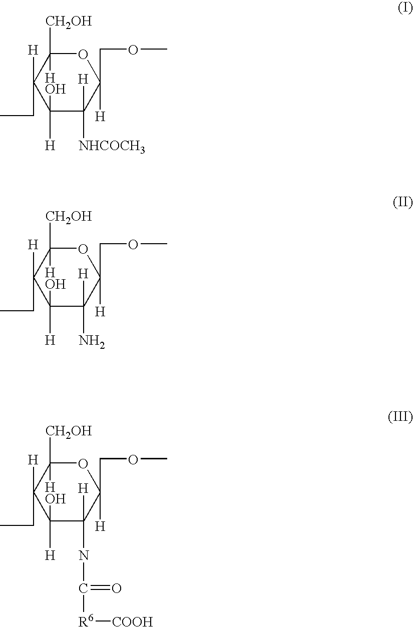
   with (I) representing from 0 to 30%, (II) from 5% to 50% and (III) from 30% to 90% in which R⁶ represents a radical of formula: 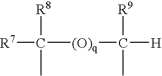
   in which q denotes 0 or 1; and if q=0, R⁷, R⁸ and R⁹, which may be identical or different, represent a hydrogen, a methyl, hydroxyl, acetoxy, amino, monoalkylamino or dialkylamino group, optionally interrupted with one or more nitrogen atoms and/or optionally substituted with one or more amine, hydroxyl or carboxyl groups, alkylthio groups optionally bearing an amino group, or sulphonic group; or, if q=1, R⁷, R⁸ and R⁹, which may be identical or different, represent a hydrogen, and also the salts formed by these compounds with acids or bases;
(6) Polymers derived from the N-carboxyalkylation of chitosan, for instance N-carboxymethylchitosan or N-carboxybutylchitosan (Evalsan from Jan Dekker);
(7) Polymers as described in FR 1 400 366: 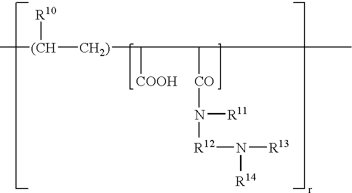
   in which R¹⁰ is a hydrogen, CH₃O—, CH₃CH₂O— or phenyl, R¹¹ and R¹⁴, which may be identical or different, represent a hydrogen or an alkyl radical (methyl or ethyl), R¹³ represents an alkyl radical (methyl or ethyl) or a radical of formula —R¹²—N(R¹⁴)₂, R¹² representing —(CH₂)₂—, —(CH₂)₃— or —CH₂—CH(CH₃)— and also the higher homologues of these radicals and containing up to 6 carbon atoms, and r is such that the molecular weight is between 500 and 6 000 000 and preferably between 1 000 and 1 000 000;
(8) Amphoteric polymers of the type -D¹-X-D¹-X- chosen from:
a) polymers obtained by the action of chloroacetic acid or sodium chloroacetate on compounds comprising at least one unit of formula -D₁-X-D¹-X-D¹- in which D¹ denotes a piperazinyl radical and X denotes the symbol E or E′, E or E′, which may be identical or different, denote a divalent radical which is an alkylene radical containing a straight or branched chain containing up to 7 carbon atoms in the main chain, optionally substituted with hydroxyl groups and possibly also comprising oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;
b) polymers of formula -D¹-X-D¹-X- in which D¹ denotes a piperazinyl radical and X denotes the symbol E or E′ and at least once E′; E having the meaning given above and E′ being a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate;
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

Among the cationic or amphoteric conditioning polymers that may be used, the following are especially preferred:
(i) among the cationic polymers:
the dimethyldiallylammonium chloride homopolymer (Merquat 100 from Nalco);
copolymers of dimethyldiallylammonium chloride and of acrylamide (Merquat 2200 from Nalco);
polymers of poly(quaternary ammonium) type prepared and described in FR 2 270 846, consisting of repeating units of formulae (W) and (U) below: 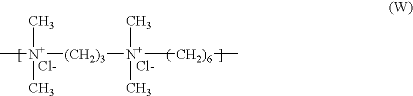
   and especially those with a molecular weight, determined by gel permeation chromatography, of between 9 500 and 9 900; 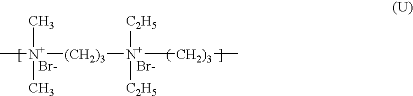
   and especially those with a molecular weight, determined by gel permeation chromatography, of about 1 200;
polymers of poly(quaternary ammonium) type of the family (11) with X⁻ denoting chlorine, and especially those with a weight-average molecular mass of less than 100 000 and preferably less than or equal to 50 000;
(ii) among the amphoteric polymers:
dimethyldiallylammonium chloride/acrylic acid copolymer (80/20) (Merquat 280 from Nalco—CTFA name: Polyquaternium 22);
dimethyldiallylammonium chloride/acrylic acid copolymer (95/5) (Merquat 295 from Nalco);
methacrylamidopropyltrimonium chloride, acrylic acid and ethyl acrylate copolymer (Merquat 2001 from Nalco—CTFA name: Polyquaternium 47);
acrylamide/dimethyldiallylammonium chloride/acrylic acid terpolymer (Merquat Plus 3330 from Nalco—CTFA name: Polyquaternium 39).

When the oxidizing composition comprises one or more cationic or amphoteric conditioning polymers, this or these polymer(s) then generally represent from 0.01% to 10% by weight and preferably from 0.05% to 5% of the total weight of this composition.

(B) Nonionic, Anionic, Cationic or Amphoteric Amphiphilic Polymers Comprising a Hydrophobic Chain:

More particularly, the hydrophobic chain is a saturated or unsaturated, aromatic or non-aromatic, linear or branched C₆-C₃₀ hydrocarbon-based chain, optionally comprising one or more oxyalkylene (oxyethylene and/or oxypropylene) units.

Among the cationic amphiphilic polymers comprising a hydrophobic chain that may be found are cationic polyurethanes or cationic copolymers comprising vinyllactam units and in particular vinylpyrrolidone units.

Preferably, the amphiphilic polymers comprising a hydrophobic chain will be of nonionic or anionic nature.

As examples of nonionic amphiphilic polymers containing a hydrophobic chain, mention may be made, inter alia, of:
(1) celluloses modified with groups comprising at least one saturated or unsaturated, linear or branched C₆-C₃₀ hydrocarbon-based chain, for instance hydroxyethylcelluloses modified with groups comprising at least one hydrophobic chain as defined previously, such as especially Natrosol Plus Grade 330 CS(C₁₆ alkyls—sold by the company Aqualon); Bermocoll EHM 100 (sold by the company Berol Nobel), Amercell Polymer HM-1500 (hydroxyethylcellulose modified with a polyethylene glycol (15) nonylphenyl ether group—sold by the company Amerchol);
(2) hydroxypropyl guars modified with groups comprising at least one hydrophobic chain as defined, for example Jaguar XC-95/3 (C₁₄ alkyl chain—sold by the company Rhodia Chimie); Esaf lor HM 22 (C₂₂ alkyl chain—sold by the company Lamberti); RE210-18 (C₁₄ alkyl chain) and RE205-1 (C₂₀ alkyl chain) sold by the company Rhodia Chimie;
(3) copolymers of vinylpyrrolidone and of hydrophobic monomers containing a hydrophobic chain as defined above, for instance Antaron or Ganex V216 (vinylpyrrolidone/hexadecene copolymers); Antaron or Ganex V220 (vinylpyrrolidone/eicosene copolymers), sold by the company I.S.P.;
(4) copolymers of C₁-C₆ alkyl (meth)acrylates and of amphiphilic monomers containing a hydrophobic chain;
(5) copolymers of hydrophilic (meth)acrylates and of hydrophobic monomers comprising at least one hydrophobic chain, for instance the polyethylene glycol methacrylate/lauryl methacrylate copolymer;
(6) polymers with an aminoplast ether skeleton containing at least one fatty chain, such as the Pure Thix compounds sold by the company Sud-Chemie;
(7) linear (block structure), grafted or starburst polyurethane polyethers comprising in their chain at least one hydrophilic block., which is generally a polyoxyethylene block which may comprise between 50 and 1 000 oxyethylene units approximately, and at least one hydrophobic block, which may comprise aliphatic groups alone, optionally combined with cycloaliphatic and/or aromatic blocks. Preferably, the polyurethane polyethers comprise at least two C₆-C₃₀ hydrocarbon-based hydrophobic chains, separated by a hydrophilic block; the hydrophobic chains may be pendent chains or chains with one or more of the end groups of the hydrophilic block(s).

The polyurethane polyethers comprise a urethane bond between the hydrophilic blocks, but may also contain hydrophilic blocks linked to the lipophilic blocks via other chemical bonds.

The polyurethane polyethers are in particular those described in the article by G. Formum, J. Bakke and Fk. Hansen—Colloid Polym. Sci. 271, 380-389 (1993). Examples of polyurethane polyethers that may be mentioned include Nuvis FX 1100 (European and US INCI name “Steareth-100/PEG-136/HMDI Copolymer” sold by the company Servo Delden); Rheolate 205, 208, 204 or 212 (sold by the company Rheox); Elfacos T210 (Cl₂-C₁₄ alkyl chain) and Elfacos T212 (C₁₈ alkyl chain) sold by the company Akzo.

The anionic amphiphilic polymers containing a hydrophobic chain that may be used comprise, as hydrophobic chain, at least one saturated or unsaturated, aromatic or non-aromatic, linear or branched C₈-C₃₀ hydrocarbon-based chain.

More particularly, the anionic amphiphilic polymers comprising at least one hydrophobic chain which are crosslinked or non-crosslinked, comprise at least one hydrophilic unit derived from one or more ethylenically unsaturated monomers bearing a carboxylic acid function, or a sulphonic function which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Anionic amphiphilic polymers of the type described above are described and prepared, for example, in patents U.S. Pat. No. 3,915,921 and U.S. Pat. No. 4,509,949 (copolymers of (meth)acrylic acid and of C₁₀-C₃₀ alkyl (meth)acrylates) or in patent EP 216 479 (copolymers of (meth)acrylic acid and of fatty alkyl allyl ethers).

The amphiphilic polymers comprising at least one sulphonic group, in free or partially or totally neutralized form and at least one hydrophobic portion are described, for example, in FR 00/16954 and FR 01/00328, the content of which forms an integral part of the present invention.

Among these, mention may be made more particularly of acrylamido-2-methyl-2-propanesulphonic (AMPS) acid/n-dodecylacrylamide copolymer neutralized with sodium hydroxide, the copolymer crosslinked with methylenebisacrylamide consisting of 75% by weight of AMPS units neutralized by NH₃ and 25% by weight of Genapol T-250 acrylate units, the copolymer crosslinked with allyl methacrylate consisting of 90% by weight of AMPS units neutralized with NH₃ and 10% by weight of Genapol T-250 methacrylate units, or the copolymer crosslinked with allyl methacrylate consisting of 80% by weight of AMPS units neutralized with NH₃ and 20% by weight of Genapol T-250 methacrylate units.

Examples of preferred polymers that may be mentioned include Carbopol ETD-2020 (acrylic acid/C₁₀-C₃₀ alkyl methacrylate crosslinked copolymer—sold by the company Noveon); Carbopol 1382, Pemulen TR1 and Pemulen TR2 (acrylic acid/C₁₀-C₃₀ alkyl acrylate crosslinked copolymers—sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (Aculyn 28 sold by Rohm & Haas) and the methacrylic acid/ethyl acrylate/steareth-10 allyl ether crosslinked copolymer.

When the oxidizing composition comprises one or more amphiphilic polymer(s) containing a hydrophobic chain, then this or these polymer(s) generally represent(s) from 0.05% to 20% by weight and better still from 0.1% to 10% of the total weight of this composition.

(C) Surfactants:

The surfactant(s) that may be present in the oxidizing composition may be chosen, without preference, from anionic, nonionic, amphoteric and cationic surfactants.

Anionic, nonionic, amphoteric or cationic surfactants that are suitable for use in the invention are especially the following:

Anionic Surfactants:

By way of examples of anionic surfactants that may be used, alone or as mixtures, mention may be made of salts, in particular alkaline salts (sodium salts, magnesium salts, ammonium salts, amine salts, amino alcohol salts, etc.) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄) alkyl sulphosuccinates, (C₆-C₂₄) alkyl ether sulphosuccinates, (C₆-C₂₄) alkylamide sulphosuccinates; (C₆-C₂₄) alkyl sulphoacetates; (C₆-C₂₄)acyl sarcosinates; and (C₆-C₂₄)acyl glutamates.

Mention may also be made of (C₆-C₂₄)alkylpolyglycoside carboxylic esters such as alkylpolyglucoside citrates, alkylpolyglucoside tartrates, alkylpolyglucoside sulphosuccinates and alkylpolyglucoside sulphosuccinamates; acyl isethionates and N-acyl taurates, the alkyl or acyl radical of all of these different compounds preferably containing from 12 to 20 carbon atoms and the aryl radical preferably denoting a phenyl or benzyl group.

It is also possible to use fatty acid salts such as the salts of oleic, ricinoleic, palmitic and stearic acid, and the salts of coconut oil acid or hydrogenated coconut oil acid; acyl lactylates in which the acyl radical contains 8 to 20 carbon atoms; alkyl D-galactoside uronic acids and their salts; polyoxyalkylenated (C₆-C₂₄)alkyl ether carboxylic acids, polyoxyalkylenated (C₆-C₂₄)alkylamido ether carboxylic acids and their salts, in particular those comprising from 2 to 50 alkylene oxide groups and more, especially ethylene oxide groups, and mixtures thereof.

Nonionic Surfactants:

Nonionic surfactants are compounds that are well known per se (see for example the “Handbook of Surfactants” by M. R. Porter, published by Blackie & Son (Glasgow and London), 1991, pp. 116-178) and their nature is not a critical feature in the context of the present invention.

Thus, used alone or as mixtures, they can be chosen especially from polyethoxylated and polypropoxylated, alkylphenols, α-diols or alcohols having a fatty chain containing, for example, 8 to 18 carbon atoms, it being possible for the number of ethylene oxide or propylene oxide groups to range in particular from 2 to 50; copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol; alkylpolyglycosides; N-alkylglucamine derivatives, and amine oxides such as (C₁₀-C₁₄)alkylamine oxides or N-acylaminopropylmorpholine oxides.

Amphoteric Surfactants:

The amphoteric (or zwitterionic) surfactants, the nature of which is not a critical feature in the context of the present invention, may be chosen especially, alone or as mixtures, from aliphatic secondary or tertiary amine derivatives in which the aliphatic radical is a linear or branched chain containing 8 to 18 carbon atoms and containing at least one water-solubilizing anionic group, for example carboxylate, sulphonate, sulphate, phosphate or phosphonate.

Mention may also be made of (C₈-C₂₀)alkylbetaines, sulphobetaines, (C₈-C₂₀) alkylamido(C₁-C₆)alkylbetaines or (C₈-C₂₀)alkylamido(C₁-C₆)alkylsulphobetaines.

Among the amine derivatives, mention may be made especially of the products sold by the company Rhodia Chimie under the trade name Miranol®, which are described in U.S. Pat. No. 2,528,378 and U.S. Pat. No. 2,781,354 and classified in the CTFA dictionary, 5th edition, 1993, under the names “disodium cocoamphodiacetate”, “disodium lauroamphodiacetate”, “disodium caprylamphodiacetate”, “disodium capryloamphodiacetate”, “disodium cocoamphodipropionate”, “disodium lauroamphodipropionate”, “disodium caprylamphodipropionate”, “disodium capryloamphodiprbpionate”, “lauroamphodipropionic acid” and “cocoamphodipropionic acid”.

Cationic Surfactants:

As cationic surfactants that may be used, alone or as mixtures, mention may be made of salts of optionally polyoxyalkylenated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; and amine oxides of cationic nature.

When the oxidizing composition comprises one or more surfactants, then this or these agent(s) generally represent(s) from 0.01% to 40% by weight and better still from 0.1% to 30% of the total weight of this composition.

(D) Rheology Modifiers Other Than the Polymers (B):

For the purposes of the present invention, the term “rheology modifier” means any compound capable of giving a viscosity to the oxidizing composition such that, once it is applied onto keratin fibres, this composition does not run, and remains perfectly localized at the point of application

It should be noted that said agent described below lacks a hydrophobic chain, i.e. a saturated or unsaturated, aromatic or non-aromatic, linear or branched C₈-C₃₀ hydrocarbon-based chain optionally comprising one or more oxyalkylene (oxyethylene and/or oxypropylene) units.

The rheology modifier(s) that may be present in the oxidizing composition is (are) polymers of natural origin or synthetic polymers, and are advantageously chosen from those conventionally used in cosmetics.

Examples of synthetic polymers that may be mentioned include polyvinylpyrrolidone, polyacrylic acid, polyacrylamide, non-crosslinked poly(2-acrylamidopropanesulphonic acid) (Simugel EG from the company SEPPIC), crosslinked poly(2-acrylamido-2-methylpropanesulphonic acid), free or partially neutralized with ammonia (Hostacerin AMPS from Clariant), mixtures of non-crosslinked poly(2-acrylamido-2-methylpropanesulphonic acid) with hydroxyalkylcellulose ethers or with poly(ethylene oxide)s, as described in patent U.S. Pat. No. 4,540,510; mixtures of poly((meth)acrylamido(C₁-C₄)alkylsulphonic acid), which is preferably crosslinked, with a crosslinked copolymer of maleic anhydride and of a (C₁-C₅)alkyl vinyl ether (Hostacerin AMPS/Stabileze QM from the company ISF).

The thickening polymers of natural origin are preferably polymers comprising at least one sugar unit, for instance nonionic guar gums, optionally modified with C₁C₆ hydroxyalkyl groups; biopolysaccharide gums of microbial origin, such as scleroglucan gum or xanthan gum; gums derived from plant exudates, such as gum arabic, ghatti gum, karaya gum, gum tragacanth, carrageenan gum, agar gum and carob gum; pectins; alginates; starches; hydroxy (C₁-C₆) alkylcelluloses and carboxy(C₁-C₆) alkylcelluloses.

It should be noted that the term “sugar unit” denotes a monosaccharide (i.e. monosaccharide or oside or simple sugar) portion, an oligosaccharide portion (short chains formed from a sequence of monosaccharide units, which may be different) or a polysaccharide portion [long chains consisting of monosaccharide units, which may be different, i.e. polyholosides or polyosides]. The saccharide units may also be substituted with alkyl, hydroxyalkyl, alkoxy, acyloxy or carboxyl radicals, the alkyl radicals containing from 1 to 4 carbon atoms.

Examples of nonionic, unmodified guar gums that may be mentioned, inter alia, include Guargel D/15 (Goodrich); Vidogum GH 175 (Unipectine), Meypro-Guar 50 and Jaguar C (Meyhall/Rhodia Chimie); and the modified nonionic guar gums that may be mentioned include Jaguar HP8, HP60, HP120, DC 293 and HP 105 (Meyhall/Rhodia Chimie); Galactasol 4H4FD2 (Aqualon).

The biopolysaccharide gums of microbial or plant origin are well known to those skilled in the art and are described especially in the book by Robert L. Davidson entitled “Handbook of Water soluble gums and resins” published by McGraw Hill Book Company (1980).

Among these gums, mention will be made of scleroglucans such as, especially, Actigum CS from Sanofi Bio Industries; Amigel from Alban Muller International, and also the glyoxal-treated scleroglucans described in FR 2 633 940); xanthan gums, for instance Keltrol, Keltrol T, Keltrol Tf, Keltrol Bt, Keltrol Rd, Keltrol Cg (Nutrasweet Kelco), Rhodicare S and Rhodicare H (Rhodia Chimie); starch derivatives, for instance Primogel (Avebe); hydroxyethylcelluloses such as Cellosize QP3L, QP4400H, QP30000H, HEC30000A and Polymer PCG10 (Amerchol), Natrosol 250HHR, 250MR, 250M, 250HHXR, 250HHX, 250HR, HX (Hercules) and Tylose H1000 (Hoechst); hydroxypropylcelluloses, for instance Klucel EF, H, LHF, MF and G (Aqualon); carboxymethylcelluloses, for instance Blanose 7M8/SF, refined 7M, 7LF, 7MF, 9M31F, 12M31XP, 12M31P, 9M31XF, 7H, 7M31, 7H3SXF (Aqualon), Aquasorb A500 (Hercules), Ambergum 1221 (Hercules), Cellogen HP810A, HP6HS9 (Montello) and Primellose (Avebe).

The composition may also comprise in replacement for or in combination with at least one rheology modifier, at least one linear or non-linear, saturated or unsaturated C₆-C₃₀ carboxylic acid alkylamide, and optionally bearing one or more hydroxyl groups.

Moreover, the nitrogen of the amide group may be monosubstituted or disubstituted. It is preferably monosubstituted.

The amide may comprise 1 to 20 oxyalkylene (oxyethylene and/or oxypropylene) units, preferably oxyethylene units.

When the oxidizing composition comprises one or more rheology modifiers, then this or these agent(s) generally represent(s) from 0.05% to 20% by weight and better still from 0.1% to 10% of the total weight of this composition.

(E) pH Modifiers:

The pH of the oxidizing composition may be between 1.5 and 12.

However, it is preferable for this pH to be between 1.5 and 10 and better still between 1.5 and 7 in the case where the oxidizing composition is intended for bleaching keratin fibres, and for it to be between 6 and 12 and preferably between 7 and 11 when it is intended for permanently reshaping keratin fibres.

Such pH values may be obtained using acidifying or basifying agents.

As examples of acidifying agents that may be used, mention may be made of mineral or organic acids, for instance hydrochloric acid, phosphoric acid, orthophosphoric acid, acetic acid, tartaric acid, citric acid, lactic acid, boric acid and sulphonic acids.

The basifying agents may themselves be chosen especially from aqueous ammonia, ammonium or alkaline carbonates, alkanolamines such as monoethanolamine, diethanolamine and triethanolamine and derivatives thereof, hydroxyalkylamines, oxyethylenated and/or oxypropylenated ethylenediamines, sodium hydroxide, potassium hydroxide and the compounds of the formula (XIX) below: 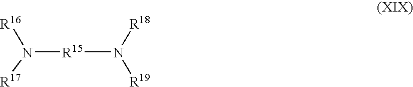
in which:
R¹⁵ is a propylene residue optionally substituted with a hydroxyl group or a C₁-C₄ alkyl radical; whereas
R¹⁶, R¹⁷, R¹⁸ and R¹⁹, which are identical or different, represent a hydrogen atom, a C₁-C₄ alkyl radical or a C₁-C₄ hydroxyalkyl radical.

When the oxidizing composition comprises one or more acidifying agents or one or more basifying agents, then this or these agent(s) generally represent(s) from 0.01% to 30% by weight relative to the total weight of this composition.

(F) Solvents:

The solvents that may be present in the oxidizing composition are especially water and mixtures composed of water and of one or more cosmetically acceptable organic solvents, this or these organic solvent(s) possibly being, in particular, alcohols such as ethanol, isopropanol, benzyl alcohol, phenylethyl alcohol or cetyl alcohol; polyols, for instance propylene glycol and glycerol; glycol ethers, for instance ethylene glycol monomethyl ether, monoethyl ether and monobutyl ether, and also glycol alkyl ethers, for instance diethylene glycol monoethyl ether or monobutyl ether.

This or these organic solvent(s), when it is (they are) present in the oxidizing composition, generally represent(s) from 0.5% to 20% by weight and better still from 2% to 10% by weight relative to the total weight of this composition.

Depending on its intended use and the particular properties desired to be given thereto, the oxidizing composition may also comprise one or more additives chosen from:
mineral or organic fillers such as silica or clays, and/or
binders such as vinylpyrrolidone, oils or waxes, polyalkylene glycols or polyalkylene glycol derivatives, and/or
lubricants such as polyol stearates or alkali metal or alkaline-earth metal stearates, and/or
antifoams, and/or
volatile or non-volatile, cyclic, linear or branched silicones, which are optionally modified, especially with amine groups, and/or
dyes, and/or
matting agents, for instance titanium oxides, and/or
preserving agents, and/or
fragrances.

Each of these adjuvants may represent, when it is present in the oxidizing composition, up to 30% by weight relative to the total weight of this composition.

In accordance with the invention, the oxidizing composition is a composition that is preferably intended for dyeing, bleaching or permanently reshaping human keratin fibres, and more especially the hair.

A subject of the present invention is also an oxidizing composition for dyeing, bleaching or permanently reshaping keratin fibres, comprising at least one oxidizing agent and at least one compound corresponding to formula (I) below:
R—N— (CH(R′)CO₂x)₂ (I)
in which:
R represents a hydrogen atom or a group CH(CO₂X)—(CH₂)₂CO₂X, CH(CH₃)—CO₂X or (CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group CH₂CO₂X if R represents a hydrogen atom, or R′ represents a hydrogen atom if R is other than a hydrogen atom; and
X represents a hydrogen atom or a monovalent or divalent cation derived from an alkali metal, from an alkaline-earth metal, from a transition metal, from an organic amine or from an ammonium cation;
   with the proviso that, when the oxidizing agent is sodium perborate, the compound of formula (I) is other than methylglycinediacetic acid and iminodisuccinic acid.

According to the invention, when X represents a monovalent metal cation, this cation may be chosen from the group of alkali metal cations, such as K⁺ and Na⁺.

According to the invention, when X represents a divalent metal cation, this cation may be chosen from the group of alkaline-earth metal cations or transition metal cations, such as Ca²⁺, Mg²⁺, Co²⁺, Fe²⁺, Mn²⁺ and Zn²⁺.

As mentioned previously, among the compounds of formula (I) above that can form part of the composition of the invention, mention may be made of the preferred compounds chosen from the group consisting of methylglycinediacetic acid, N-lauroyl-N,N′,N′-ethylenediamiaminetriacetic acid, iminodisuccinic acid, N,N-dicarboxymethyl-L-glutamic acid and iminodisuccinic acid, and the corresponding salts thereof, the compound that is most preferred being methylglycinediacetic acid.

As mentioned previously, the oxidizing composition according to the invention comprises at least one oxidizing agent, said oxidizing agent possibly being chosen from hydrogen peroxide, urea peroxide, alkali metal bromates, persalts such as perborates, percarbonates and persulphates, and peracids. The oxidizing agent is preferably a persulphate.

In accordance with the invention, the compositions that are preferred are those in which the compounds of formula (I) are methylglycinediacetic acid or iminodisuccinic acid, optionally in the form of salts, and the oxidizing agent is aqueous hydrogen peroxide solution.

As mentioned previously, the compound(s) of formula (I) preferably represent(s) from 0.001% to 5% by weight relative to the total weight of the oxidizing composition.

As mentioned previously, the oxidizing composition according to the invention may comprise, in addition to the compound(s) of formula (I) and the oxidizing agent(s), one or more compounds chosen from:
cationic or amphoteric substantive polymers (A);
nonionic, anionic, cationic or amphoteric amphiphilic polymers comprising at least one fatty chain (B);
anionic, nonionic, cationic, amphoteric or zwitterionic surfactants (C);
rheology modifiers (D) other than the polymers (B);
pH modifiers (E);
solvents (F);
other adjuvants.

These compounds may be chosen from those mentioned previously, and are present in the oxidizing composition in proportions similar to those mentioned previously for each of them.

A subject of the present invention is also a process for bleaching or permanently reshaping keratin fibres, successively comprising the steps consisting in:
a) applying an oxidizing composition as defined above to keratin fibres;
b) leaving the oxidizing composition to stand on the keratin fibres for a time that is sufficient to obtain the desired bleaching or permanent reshaping;
c) rinsing the keratin fibres to remove the oxidizing composition therefrom;
d) optionally washing the keratin fibres one or more times, rinsing them after each wash, and optionally drying them;
   said process also comprising, before step a), in the case of a permanent reshaping, the steps consisting in:
i) applying a reducing composition to the keratin fibres, said keratin fibres being placed under mechanical tension before, during or after said application;
ii) leaving the reducing composition to stand on the keratin fibres for a time that is sufficient to obtain the desired reshaping; and
iii) optionally rinsing the keratin fibres with water to remove the reducing composition therefrom.

When this process is a permanent-reshaping process, the first step of this process consists in applying a reducing composition to the hair. This application is performed lock by lock or all at once.

The reducing composition comprises at least one reducing agent, which may be chosen in particular from thioglycolic acid, cysteine, cysteamine, glyceryl thioglycolate, thiolactic acid, or thiolactic acid or thioglycolic acid salts.

The usual step for placing the hair under tension in a shape corresponding to the final shape desired for this hair (for example curls) may be performed by any means, especially mechanical means, which is suitable and known per se for holding the hair under tension, for instance rollers, curlers and the like.

Before performing the optional rinsing step (iii), the head of hair onto which the reducing composition has been applied should conventionally be left to stand for a few minutes, generally between 5 minutes and 1 hour and preferably between 10 and 30 minutes, so as to give the reducing agent enough time to act correctly on the hair. This waiting phase preferably takes place at a temperature ranging from 35° C. to 45° C., while preferably also protecting the hair with a bonnet.

In the optional rinsing step, the hair impregnated with the reducing composition is then rinsed thoroughly with an aqueous composition.

Next, after the optional rinsing step, the oxidizing composition of the invention is applied to the hair (step a)), with the aim of fixing the new shape given to the hair.

As in the case of the application of the reducing composition, the head of hair onto which the oxidizing composition has been applied is then, conventionally, left in a standing or waiting phase that lasts a few minutes, generally between 3 and 30 minutes and preferably between 5 and 15 minutes.

If the tension of the hair is maintained by external means, these means (rollers, curlers and the like) may be removed from the head of hair before or after the fixing step.

Lastly, in the final step of the process according to the invention (step c), the hair impregnated with the oxidizing composition is rinsed thoroughly, generally with water.

When the process is a process for bleaching keratin fibres, said process comprises, as defined above, a step of applying an oxidizing composition according to the invention to the keratin fibres. Conventionally, a second step of the bleaching process according to the invention is a step of rinsing the keratin fibres.

A subject of the present invention is also a process for dyeing keratin fibres, successively comprising the steps consisting in:
e) applying a dye composition to these fibres;
f) developing the colour of said composition by applying to the fibres an oxidizing composition as defined above;
g) leaving the oxidizing composition to stand on the keratin fibres for a time that is sufficient to obtain the desired coloration;
h) rinsing the keratin fibres with water to remove the dye composition and the oxidizing composition therefrom.

According to another variant of the invention, the process successively comprises the steps consisting in:
i) applying to the keratin fibres a composition obtained by extemporaneous mixing, before application, of a dye composition and of an oxidizing composition as described previously;
j) leaving the composition to stand on the keratin fibres for a time that is sufficient to obtain the desired coloration;
k) rinsing the keratin fibres with water to remove the composition therefrom.

According to another particular embodiment of the invention, the dyeing process may comprise a preliminary step that consists in mixing, before application to the keratin fibres, a composition comprising at least one dye precursor with an oxidizing composition according to the invention, said oxidizing composition allowing, by virtue of the presence of an oxidizing agent and the presence of a complexing agent in accordance with the invention, the development of the dye precursor of the first composition, said resulting composition then being applied to the keratin fibres.

Irrespective of the embodiment of this dyeing process, the time required for the development generally ranges between 3 and 60 minutes and more specifically between 5 and 40 minutes, the standing time after application of the compositions to the keratin fibres ranging from 5 minutes to 1 hour and preferably from 10 to 30 minutes.

Another subject of the present invention is a device or “kit” for dyeing keratin fibres, comprising at least two compositions A and B intended to be mixed together to obtain a ready-to-use dye composition, the composition A being the oxidizing composition and the composition B being a composition comprising at least one dye, said device being characterized in that the composition A contains at least one or more compounds corresponding to the general formula (I) below:
R—N—(CH(R′)CO₂X)₂ (I)
in which:
R represents a hydrogen atom or a group CH(CO₂X)—(CH₂)₂CO₂X, CH(CH₃)—CO₂X or (CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group CH₂CO₂X when R represents a hydrogen atom, or R′ represents a hydrogen atom when R is other than a hydrogen atom; and
X represents a hydrogen atom or a monovalent or divalent cation derived from an alkali metal, from an alkaline-earth metal, from a transition metal or from an organic amine, or an ammonium cation; with the proviso that, when the oxidizing agent is sodium perborate, the compound of formula (I) is other than methylglycinediacetic acid and iminodisuccinic acid.

A subject of the invention is also a device or “kit” for bleaching keratin fibres, comprising at least two compositions C and D intended to be mixed together to obtain a ready-to-use oxidizing composition, said device being characterized in that at least one of the compositions C and D contains one or more oxidizing agents and at least one contains one or more compounds corresponding to the general formula (I) below:
R—N—(CH(R′) CO₂X)₂ (I)
in which:
R represents a hydrogen atom or a group CH(CO₂X)—(CH₂)₂CO₂X, CH(CH₃)—CO₂X or (CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group CH₂CO₂X when R represents a hydrogen atom, or R′ represents a hydrogen atom when R is other than a hydrogen atom; and
X represents a hydrogen atom or a monovalent or divalent cation derived from an alkali metal, from an alkaline-earth metal, from a transition metal or from an organic amine, or an ammonium cation;
   with the proviso that, when the oxidizing agent is sodium perborate, the compound of formula (I) is other than methylglycinediacetic acid and iminodisuccinic acid.

Finally, a subject of the invention is a device or “kit” for permanently reshaping keratin fibres, comprising at least two compositions E and F, composition E being an oxidizing composition and composition F being a reducing composition, said device being characterized in that composition E contains one or more oxidizing agents and at least one or more compounds corresponding to the general formula (I) below:
R—N—(CH(R′) CO₂X)₂ (I)
in which:
R represents a hydrogen atom or a group CH(CO₂X) (CH₂)₂CO₂X, CH(CH₃)—CO₂X or (CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group CH₂CO₂X when R represents a hydrogen atom, or R′ represents a hydrogen atom when R is other than a hydrogen atom; and
X represents a hydrogen atom or a monovalent or divalent cation derived from an alkali metal, from an alkaline-earth metal, from a transition metal or from an organic amine, or an ammonium cation;
   with the proviso that, when the oxidizing agent is sodium perborate, the compound of formula (I) is other than methylglycinediacetic acid and iminodisuccinic acid.

Finally, a subject of the invention is the use of a composition as defined above, or of a process as defined above or of a device as defined above, for dyeing, bleaching or permanently reshaping human keratin fibres and more especially the hair.

Besides the preceding arrangements, the invention also comprises other arrangements which will emerge from the rest of the description that follows, which refer to embodiments of reducing compositions for dyeing, bleaching and permanently reshaping keratin fibres.

It goes without saying that these examples are given for illustrative purposes and do not in any way limit the subject of the invention.

### DETAILED DESCRIPTION OF SPECIFIC EMBODIMENTS

### EXAMPLE 1

In this example, the stability of two oxidizing compositions—A and B, respectively—is tested, the oxidizing composition A being a composition comprising a complexing agent not in accordance with the invention (diethylenetriaminepentaacetic acid in salt form) and composition B being a composition comprising a complexing agent in accordance with the present invention (methylglycinediacetic acid in salt form). Table I below specifies the constituents included in compositions A and B, the amounts being expressed as percentages by mass.

TABLE I

| Constituents | Composition A | Composition B |
|---|---|---|
| Diethylenetriamine- | 0.176 | — |
| pentaacetic acid in | (i.e. 0.14 mol) | |
| the form of the | | |
| pentasodium salt, | | |
| as an aqueous 40% | | |
| solution⁽¹⁾ | | |
| Methylglycinediacetic | — | 0.075 |
| acid in the | | (i.e. 0.14 mol) |
| form of the | | |
| trisodium salt, as | | |
| an aqueous 40% | | |
| solution⁽²⁾ | | |
| 50% aqueous | 12 | 12 |
| hydrogen peroxide | | |
| solution | | |
| 85% phosphoric acid | qs pH = 2 | qs pH = 2 |
| Water | qs 100 g | qs 100 g |

⁽¹⁾Dissolvine ® from Azko
⁽²⁾Trilon M Liquide ® from BASF

The stability of these two compositions is determined by measuring the aqueous hydrogen peroxide titre, before (t1) and after (t2).

The stability index corresponds to the ratio of the aqueous hydrogen peroxide titre at t2 to the aqueous hydrogen peroxide titre at t1.

Three tests were performed for each composition. Table 2 collates the mean stability indices for each of the abovementioned compositions.

TABLE II

| Stability of the | | |
|---|---|---|
| oxidizing | | |
| compositions | Composition A | Composition B |
| Residual H₂O₂ | | |
| content: | | |
| Mean | 96.4% | 99.4% |
| (Standard | (0.7%) | (0.5%) |
| deviation) | | |
| Volume O₂ released | 151 ml | 23 ml |
| per 100 g of | | |
| composition: | | |

### EXAMPLE 2

In this example, two oxidizing compositions—C and D, respectively—intended for use in dyeing keratin fibres were prepared, composition C being a composition comprising a complexing agent not in accordance with the invention, and composition D being a composition comprising a complexing agent in accordance with the invention.

Table III represents the qualitative and quantitative compositions of these compositions, the amounts being expressed as percentages by mass.

TABLE III

| Constituents | Composition C | Composition D |
|---|---|---|
| Diethylenetriamine- | 0.150 | — |
| pentaacetic acid in | (i.e. 0.12 mmol) | |
| the form of the | | |
| pentasodium salt, | | |
| as an aqueous 40% | | |
| solution⁽⁹⁾ | | |
| Methylglycinediacetic | — | 0.065 |
| acid in the | | (i.e. 0.12 mmol) |
| form of the | | |
| trisodium salt, as | | |
| an aqueous 40% | | |
| solution⁽¹⁰⁾ | | |
| Sodium salicylate | 0.024 | 0.024 |
| Tetrasodium | 0.01 | 0.01 |
| pyrophosphate | | |
| decahydrate | | |
| 50% aqueous | 12 | 12 |
| hydrogen peroxide | | |
| solution | | |
| 85% phosphoric acid | qs pH = 2 | qs pH = 2 |
| Water | qs 100 g | qs 100 g |

⁽⁹⁾Dissolvine ® from Akzo
⁽¹⁰⁾Trilon M Liquide ® from BASF

Compositions C and D were mixed with an equal amount of a neutral dye composition Recital, into which is introduced:
10⁻³ mol of p-aminophenol, and
−10⁻³ mol of 4-amino-2-hydroxytoluene.

The resulting compositions are applied for 30 minutes to locks of permanent-waved natural grey hair containing 90% white hairs, in a proportion of 10 g of composition per 1 g of lock of hair.

### EXAMPLE 3

In this example, an oxidizing composition E intended for use in permanently reshaping keratin fibres, and comprising a complexing agent in accordance with the invention, was prepared.

Table IV gives its qualitative and quantitative composition, the amounts being expressed in percentages by mass.

TABLE IV

| Constituents | Composition E |
|---|---|
| Cetyl alcohol | 3 |
| Sodium lauryl sulphate | 0.5 |
| Polyglycerolated (2 mol) | 0.45 |
| oleyl alcohol | |
| Polyglycerolated (4 mol) | 0.35 |
| oleyl alcohol | |
| Simethicone | 0.045 |
| Methylglycinediacetic acid | 0.13 |
| in the form of the | |
| trisodium salt, as an | |
| aqueous 40% solution | |
| Tetrasodium pyrophosphate | 0.02 |
| decahydrate | |
| Sodium salicylate | — |
| Sodium stannate hexahydrate | 0.04 |
| 50% aqueous hydrogen | 24 |
| peroxide solution | |
| Aqueous 85% phosphoric acid | qs pH = 2 |
| solution | |
| Water | qs 100 g |

### EXAMPLE 4

In this example, two oxidizing compositions—F and G, respectively—intended for use in bleaching were prepared, these compositions both being anhydrous compositions, in pulverulent form, comprising a complexing agent in accordance with the invention.

Table V shows the qualitative and quantitative compositions of these compositions, the amounts being expressed as percentages by mass.

TABLE V

| Constituents | Composition F | Composition G |
|---|---|---|
| Potassium persulphate | 39.5 | 46 |
| Sodium persulphate | 30 | 15 |
| Sodium disilicate | — | 15 |
| Sodium metasilicate | 14 | 4 |
| Ammonium chloride | 6 | 4 |
| Urea | — | 4.5 |
| Sodium iminodisuccinate⁽¹³⁾ | 1 | 0.8 |
| Hexamethyl diisocyanate/ | 0.5 | — |
| polyethylene glycol | | |
| copolymer containing α and | | |
| ω stearyl polyoxyethylene | | |
| end groups⁽¹⁴⁾ | | |
| Acrylic acid/(C10/C30) | — | 1 |
| alkyl methacrylate crosslinked | | |
| copolymer⁽¹⁵⁾ | | |
| Potato carboxymethyl | — | 2 |
| starch/weakly crosslinked | | |
| sodium salt | | |
| Guar gum | 2 | 1.5 |
| Dye (ultramarine) | 0.5 | — |
| Titanium oxide | 0.5 | 1 |
| Sodium lauryl sulphate | 2 | 2 |
| Calcium stearate | 1 | 1 |
| Fumed silica of | 3 | 0.2 |
| hydrophilic nature | | |
| Hydrogenated polydecene⁽¹⁶⁾ | — | 2 |

⁽¹³⁾Imino disuccinate VP OC sodium salt powder (N-305) from Bayer;
⁽¹⁴⁾SER-AD FX 1100 ® from Servo Delden;
⁽¹⁵⁾Carbopol ETD 2020 ® from Noveon;
⁽¹⁶⁾Silkflo 366 NF Polydecene ® from Amoco Chemical

The bleaching composition F (40 g) is mixed with the oxidizing composition based on aqueous hydrogen peroxide solution E (80 g). The ready-to-use bleaching mixture thus obtained is applied for 45 minutes, under a hood, to dark natural hair, and is then rinsed out thoroughly with water. After these operations, strong, uniform bleaching is obtained.

The bleaching composition G (40 g) is mixed with the oxidizing composition based on aqueous hydrogen peroxide solution E (60 g). The ready-to-use bleaching mixture thus obtained is applied for 30 minutes, under a hood, to dark natural hair, and is then rinsed out thoroughly with water. After these operations, strong, uniform bleaching is obtained, and the hair is soft, shiny and easy to disentangle.

### EXAMPLE 5

In this example, two oxidizing compositions—H and I, respectively—intended for use in bleaching were prepared, these compositions both being anhydrous compositions, in paste form, comprising a complexing agent in accordance with the invention.

Table VI shows the qualitative and quantitative compositions of these compositions, the amounts being expressed as percentages by mass.

TABLE VI

| Constituents | Composition H | Composition I |
|---|---|---|
| Potassium persulphate | 35.8 | 35.6 |
| Sodium persulphate | 6 | 6 |
| Sodium disilicate | 15 | 15 |
| Sodium metasilicate | 3 | 3 |
| Ammonium chloride | 4.2 | 4.2 |
| Sodium iminodisuccinate⁽¹⁷⁾ | 1 | 1 |
| Hexamethyl diisocyanate/ | 2 | 0.5 |
| polyethylene glycol | | |
| copolymer containing α and | | |
| ω stearyl polyoxyethylene | | |
| end groups⁽¹⁸⁾ | | |
| Acrylic acid/(C10/C30) | — | 0.5 |
| alkyl methacrylate crosslinked | | |
| copolymer⁽¹⁹⁾ | | |
| Potato carboxymethyl | 2 | 1 |
| starch/weakly crosslinked | | |
| sodium salt | | |
| Guar gum | — | 2 |
| Dye (ultramarine) | 0.5 | 0.5 |
| Titanium oxide | 1 | 1 |
| Sodium lauryl sulphate | 3.5 | 3.5 |
| Calcium stearate | 2 | 2 |
| Fumed silica of | 0.5 | 0.5 |
| hydrophilic nature | | |
| Isopropyl palmitate | 22.5 | — |
| Beeswax | 1 | — |
| Hydrogenated polydecene | — | 23 |
| Fumed silica of | — | 0.7 |
| hydrophobic nature | | |

⁽¹⁷⁾Imino disuccinate VP OC sodium salt powder (N-305) from Bayer;
⁽¹⁸⁾SER-AD FX 1100 ® from Servo Delden;
⁽¹⁹⁾Carbopol ETD 2020 ® from Noveon.

The bleaching composition H (40 g) is mixed with the oxidizing composition based on aqueous hydrogen peroxide solution E (80 g). The ready-to-use bleaching mixture thus obtained is applied for 45 minutes, under a hood, to dark natural hair, and is then rinsed out thoroughly with water. After these operations, strong uniform bleaching is obtained, and the hair is soft, shiny and easy to disentangle.

The bleaching composition I (40 g) is mixed with the oxidizing composition based on aqueous hydrogen peroxide solution E (60 g). The ready-to-use bleaching mixture thus obtained is applied for 25 minutes, under a hood, to dark natural hair, and is then rinsed out thoroughly with water. After these operations, strong, uniform bleaching is obtained, and the hair is soft, shiny and easy to disentangle.

### EXAMPLE 6

In this example, in a first stage, a reducing composition is prepared.

Table VII gives the qualitative and quantitative composition, the amounts being expressed as percentages by mass.

TABLE VII

| Constituents | Composition J |
|---|---|
| Thioglycolic acid | 9.2 |
| Arginine | 15 |
| 20% aqueous ammonia | 1.86 |
| Ammonium carbonate | 4.5 |
| Cocoylamidopropylbetaine/glyceryl mono- | 1.3 |
| laurate (25/5) as an aqueous 30% solution | |
| Peptizer | 0.8 |
| Isostearyl alcohol | 12 |
| Complexing agent | 0.4 |
| Fragrance | 0.4 |
| Water | qs 100 g |

Composition J is applied to a lock of wet hair, which has been rolled up beforehand on a curler 9 mm in diameter, the leave-in time being 10 minutes. Next, the lock thus treated is rinsed thoroughly with water.

In a second stage, an oxidizing composition, referred to as composition K, is prepared. Table VIII shows the qualitative and quantitative composition, the amounts being expressed as percentages by mass.

TABLE VIII

| Constituents | Composition K |
|---|---|
| Cetyl alcohol | 3 |
| Sodium lauryl sulphate | 0.5 |
| Polyglycerolated oleyl alcohol (2 mol) | 0.45 |
| Polyglycerolated oleyl alcohol (4 mol) | 0.35 |
| Simethicone | 0.045 |
| Sodium iminodisuccinate | 0.05 |
| Tetrasodium pyrophosphate decahydrate | 0.02 |
| Sodium salicylate | — |
| Sodium stannate hexahydrate | 0.04 |
| 50% aqueous hydrogen peroxide solution | 5.4 |
| Aqueous 85% phosphoric acid solution | qs pH = 2 |
| Water | qs 100 g |

⁽²⁰⁾XUS-40855.00 ® from Dow Chemical

Composition K is applied to the locks previously treated with the reducing composition J, by applying said composition K for a leave-in time of 10 minutes. Next, the locks are rinsed thoroughly with water. Finally, the hair is unrolled from the roller and then dried. The locks thus treated are wavy.

1. A method of complexing metal cations present in either an oxidizing composition for bleaching, dying or permanently reshaping keratin fibres, or keratin fibres, wherein said oxidizing composition comprises at least one oxidizing agent, comprising the step of:
mixing said oxidizing composition with at least one compound of formula (I):
R—N—(CH(R′)CO₂X)₂ (I)
wherein:
R is a hydrogen atom or a CH(CO₂X)—(CH₂)₂CO₂X, CH(CH₃)—CO₂X or (CH₂)₂—N(COR″)—CH₂—CO₂X group;
R″is a linear or branched alkyl group containing from 1 to 30 carbon atoms, or a cycloalkyl group containing from 3 to 30 carbon atoms;
R′ is a group CH₂CO₂X group when R is a hydrogen atom, or R′ is a hydrogen atom when R is other than a hydrogen atom; and
X is a hydrogen atom or a monovalent or divalent cation derived from an alkali metal, from an alkaline-earth metal, a transition metal, an organic amine, or an ammonium cation.

2. The method of claim 1, wherein said monovalent or divalent cation is preferably chosen from a monovalent alkali metal cations, a divalent alkaline-earth metal cation, a divalent transition metal cations or a monovalent cation derived from an organic amines or an ammonium cation.

3. The method of claim 1, wherein said divalent cation is chosen from an alkaline-earth metal or a transition metal cation.

4. The method of claim 1, wherein said oxidizing agent is hydrogen peroxide, an alkali metal bromate and persalt.

5. The method of claim 1, wherein said compound of formula (I) is methylglycinediacetic acid, N-lauroyl-N,N′,N′-ethylenediamiaminetriacetic acid, iminodisuccinic acid, N,N-dicarboxymethyl-L-glutamic acid, or a corresponding salts thereof.

6. The method of claim 1, wherein said oxidizing agent is aqueous hydrogen peroxide solution and the compound of formula (I) is methylglycinediacetic acid, optionally in the form of salts.

7. The method of claim 1, wherein said oxidizing agent is aqueous hydrogen peroxide solution and said compound of formula (I) is iminodisuccinic acid or a salt thereof.

8. The method of claim 1, wherein said composition further comprises cationic or amphoteric conditioning polymer.

9. The method of claim 1, wherein said composition further comprises an amphiphilic polymer, which is nonionic, anionic or cationic, wherein said amphiphilic polymer comprises a hydrophobic chain.

10. The method of claim 1, wherein said composition further comprises a surfactant.

11. The method of claim 1, wherein said composition further comprises a rheology modifiers other than said amphiphilic polymer of claim 9.

12. The method of claim 1, wherein said composition further comprises an acidifying or basifying agent.

13. The method of claim 1, wherein said composition further comprises a solvent chosen from water or a mixtures composed of water and a cosmetically acceptable organic solvent.

14. The method of claim 1, wherein said composition further comprises an adjuvant chosen from a mineral or organic filler, a binder, a lubricant, an antifoam, a silicone, a dye, a matting agent, a preserving agents or a fragrance.

15. An oxidizing composition for dyeing, bleaching or permanently reshaping keratin fibres, comprising:
a) at least one oxidizing agent, and
b) at least one compound of formula (I):
R—N—(CH(R′)CO₂X)_{2tm (I)}
wherein:
R is a hydrogen atom or a CH(CO₂X)—(CH₂)₂CO₂X, CH(CH₃)—CO₂X or (CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a H₂CO₂X group when R a hydrogen atom, or a hydrogen atom when R is other than a hydrogen atom; and
X is a hydrogen atom or a monovalent or divalent cation chosen from an alkali metal, alkaline-earth metal, transition metal, organic amine or an ammonium cation;
with the proviso that,
when said oxidizing agent is sodium perborate, said compound of formula (I) is other than methylglycinediacetic acid or iminodisuccinic acid.

16. The composition of claim 15, wherein said monovalent cation is an alkali metal cations.

17. The composition of claim 15, in wherein said divalent cation is an alkaline-earth metal or a transition metal cation.

18. The composition of claim 15, wherein said oxidizing agent is hydrogen peroxide, alkali metal bromate or persalt.

19. The composition of claim 18, wherein said oxidizing agent is persulphate.

20. The composition of claim 15, wherein said compound of formula (I) is methylglycinediacetic acid, N-lauroyl-N,N′,N-ethylenediamiaminetriacetic acid, N,N-dicarboxymethyl-L-glutamic acid or iminodisuccinic acid, or a salts thereof.

21. The composition of claim 15, wherein said Rsaid oxidizing agent is aqueous hydrogen peroxide solution and said compound of formula (I) is methylglycinediacetic acid or a salt thereof.

22. The composition of claim 15, further comprising at least one compound selected from the group consisting of:
a) a cationic or amphoteric conditioning polymers,
b) an amphiphilic polymer which is nonionic, anionic, cationic, or amphoteric, wherein said amphiphilic polymer comprises a hydrophobic chain,
c) a surfactant,
d) a rheology modifiers other than the amphiphilic polymer of (b),
e) an acidifying or basifying agent, and
f) a solvent.

23. The composition of claim 22, further comprising an adjuvants chosen from a mineral or organic filler, a binder, a lubricant, an antifoam, a silicone, a dye, a matting agent, a preserving agent or a fragrances.

24. A method of bleaching keratin fibres comprising the steps of:
a) applying to said keratin fibres the oxidizing composition of claim 15;
b) leaving said oxidizing composition on the keratin fibres for a sufficient time to obtain the desired bleaching;
c) rinsing said keratin fibres to remove said oxidizing composition therefrom.

25. A method of dyeing keratin fibres comprising the steps of:
a) applying a dye composition to said keratin fibres;
b) applying the oxidizing composition of claim 15 to develop the color;
c) leaving said oxidizing composition said keratin fibres for a sufficient time to obtain the desired coloration;
d) rinsing said keratin fibres with water to remove said dye composition and said oxidizing composition therefrom.

26. A method of dyeing keratin fibres comprising the steps consisting of:
a) mixing a dye composition and the oxidizing composition of claim 15 to create a mixture;
b) applying said mixture to said keratin fibres;
c) leaving said mixture said keratin fibres for a sufficient time to obtain the desired coloration;
d) rinsing said keratin fibres with water to remove said mixture therefrom.

27. A kit for dyeing keratin fibres comprising: at least two compositions A and B intended to be mixed together to obtain a ready-to-use dye composition, wherein
a) said composition A is an oxidizing composition comprising at least one compounds of formula (I):
R—N—(CH(R′)CO₂X)₂ (I)
wherein:
R is a hydrogen atom or a CH(CO₂X)—(CH₂)₂CO₂X, CH(CH₃)—CO₂X or (CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a CH₂CO₂X group when R is a hydrogen atom, or a hydrogen atom when R is other than a hydrogen atom; and
X is a hydrogen atom or a monovalent or divalent cation chosen from an alkali metal, an alkaline-earth metal, a transition metal, an organic amine or an ammonium cation;
with the proviso that,
when said oxidizing agent is sodium perborate, the compound of formula (I) is other than methylglycinediacetic acid or iminodisuccinic acid, and
b) said composition B is a composition comprising at least one dye.

28. A kit for bleaching keratin fibres comprising: at least two compositions C and D intended to be mixed together to obtain a ready-to-use oxidizing composition, wherein
a) at least one of the compositions C and D contains at least one oxidizing agent, and
b) at least one of said compositions C and D contains at least one compound of formula (I) below:
R—N—(CH(R′)CO₂X)₂ (I)
wherein:
R is a hydrogen atom or a CH(CO₂X)—(CH₂)₂CO₂X, CH(CH₃)—CO₂X or (CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a CH₂CO₂X group when R is a hydrogen atom, or a hydrogen atom when R is other than a hydrogen atom; and
X is a hydrogen atom or a monovalent or divalent cation chosen from an alkali metal, an alkaline-earth metal, a transition metal, an organic amine or an ammonium cation;
with the proviso that,
when said oxidizing agent is sodium perborate, said compound of formula (I) is other than methylglycinediacetic acid or iminodisuccinic acid.

29. A kit for permanently reshaping keratin fibres, comprising at least two compositions E and F, wherein said composition E is an oxidizing composition and said composition F is a reducing composition, wherein said composition E contains at least one oxidizing agents and at least one compound of formula (I):
R—N—(CH(R′) CO₂X)₂ (I)
wherein:
R is a hydrogen atom or a CH(CO₂X)—(CH₂)₂CO₂X, CH(CH₃)—CO₂X or (CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a CH₂CO₂X group when R is a hydrogen atom, or a hydrogen atom when R is other than a hydrogen atom; and
X is a hydrogen atom or a monovalent or divalent cation chosen from an alkali metal, an alkaline-earth metal, a transition metal, an organic amine or an ammonium cation;
with the proviso that, when said oxidizing agent is sodium perborate, said compound of formula (I) is other than methylglycinediacetic acid or iminodisuccinic acid.

30. (canceled)

31. The composition of claim 15, further comprising a cationic or amphoteric conditioning polymer.

32. The composition of claim 31, wherein said cationic or amphoteric conditioning polymer is present in an amount of from 0.01% to 10% by weight relative to the total weight of said composition.

33. The composition of claim 32, wherein said cationic or amphoteric conditioning polymer is present in an amount of from 0.05% to 5% by weight relative to the total weight of said composition.

34. The composition of claim 15, further comprising an amphiphilic polymer which is nonionic, anionic, cationic, or amphoteric, wherein said amphiphilic polymer contains a hydrophobic chain.

35. The composition of claim 34, wherein said amphiphilic polymer is present in an amount of from 0.05% to 20% by weight relative to the total weight of said composition.

36. The composition of claim 35, wherein said amphiphilic polymer is present in an amount of from 0.1% to 10% by weight relative to the total weight of said composition.

37. The composition of claim 15, further comprising a surfactant.

38. The composition of claim 37, wherein said surfactant is present in an amount of from 0.01% to 40% by weight relative to the total weight of said composition.

39. The composition of claim 19, wherein said surfactant is present in an amount of from 0.1% to 30% by weight relative to the total weight of said composition.

40. The composition of claim 15, further comprising a rheology modifier.

41. The composition of claim 40, wherein said rheology modifier is present in an amount of from 0.05% to 20% by weight relative to the total weight of said composition.

42. The composition of claim 41, wherein said rheology modifier is present in an amount of from 0.01% to 30% by weight relative to the total weight of said composition.

43. The composition of claim 15, further comprising a solvent.

44. The composition of claim 43, wherein said solvent is water or a mixture composed of water and a cosmetically acceptable organic solvent.

45. The composition of claim 43, wherein said solvent is present in an amount from 0.5% to 20% by weight relative to the total weight of said composition.

46. The composition of claim 45, wherein said solvent is present in an amount of from 2% to 10% by weight relative to the total weight of said composition.

47. The composition of claim 15, further comprising an adjuvant chosen from a mineral or organic filler, a binder, a lubricant, an antifoam, a silicone, a dye, a matting agent, a preserving agent or a fragrance.

48. The method of 24, further comprising the step of washing said keratin fibres one or more time, rinsing said keratin fibres after each wash.

49. The method of 48, further comprising the step of drying said keratin fibres.

50. A method of permanently reshaping keratin fibres, comprising the step of:
a) applying to said keratin fibres a reducing composition;
b) leaving said reducing composition on said keratin fibres for a sufficient time to obtain the desired reshaping;
c) rinsing said keratin fibres to remove said reducing composition therefrom;
d) applying the oxidizing composition of claim 15 to said keratin fibres;
e) leaving said oxidizing composition on said keratin fibres for a sufficient time to obtain the desired reshaping;
f) rinsing said keratin fibres with water to remove said oxidizing composition therefrom;
g) washing said keratin fibres one or more times, rinsing them after each wash.

51. The method of claim 50, further comprising the step of drying said keratin fibres.

52. The method of claim 8, wherein said cationic or amphoteric conditioning polymer is present in an amount from 0.01% to 10% by weight relative to the total weight of said composition.

53. The method of claim 52, wherein said cationic or amphoteric conditioning polymer is present in an amount from 0.05% to 5% by weight relative to the total weight of said composition.

54. The method of claim 9, wherein said amphiphilic polymer is present in an amount from 0.05% to 20% by weight relative to the total weight of said composition.

55. The method of claim 54, wherein said amphiphilic polymer is present in an amount from 0.1% to 10% by weight relative to the total weight of said composition.

56. The method of claim 10, wherein said surfactant is present in an amount from 0.01% to 40% by weight relative to the total weight of said composition.

57. The method of claim 56, wherein said surfactant is present in an amount from 0.1% to 30% by weight relative to the total weight of said composition.

58. The method of claim 11, wherein said surfactant is present in an amount from 0.05% to 20% by weight relative to the total weight of said composition.

59. The method of claim 58, wherein said surfactant is present in an amount from 0.1% to 10% by weight relative to the total weight of said composition.

60. The method of claim 12, wherein said acidifying or basifying agent is present in an amount from 0.01% to 30% by weight relative to the total weight of said composition.

61. The method of claim 13, wherein said solvent is present in an amount from 0.5% to 20% by weight relative to the total weight of said composition.

62. The method of claim 61, wherein said solvent is present in an amount from 2% to 10% by weight relative to the total weight of said composition.

63. The method of claim 1, further comprising a step of applying said oxidizing composition to said keratin fibres.
